(19) United States
(12) Patent Application Publication
(10) Pub. No.: US 2004/0158226 A1
(43) Pub. Date: Aug. 12, 2004

(54) Syringe tissue sieve

(75) Inventors: William Soo Hoo, Carlsbad, CA (US); Norbert Hagen, Carlsbad, CA (US); Brad Styer, San Diego, CA (US); Eugene Tu, San Diego, CA (US)

Correspondence Address: O'MELVENY & MEYERS, 114 PACIFICA, SUITE 100, IRVINE, CA, 92618, US

(73) Assignee: Genoptix, Inc.

(21) Appl. No.: 10/359,830

(22) Filed: Feb. 7, 2003

(51) Int. Cl. C12Q 1/00; C12M 1/00

(52) U.S. Cl. 604/500; 435/293.1

(57) ABSTRACT

A syringe tissue sieve for producing viable, single cell suspensions includes a syringe barrel having a lumen between a proximal end and a distal end thereof, a wire mesh screen disposed within the lumen of the syringe barrel, the wire mesh screen being oriented perpendicular to the axial direction of the lumen, and a plunger moveable within the lumen of the syringe barrel. In an alternative embodiment, the wire mesh screen is located in a collar assembly which can be loaded into off-the-shelf syringes.

### FIELD OF THE INVENTION

The field of the invention generally relates to devices and methods for obtaining viable, single cell suspensions from tissue samples. In particular, the field of the invention relates to tissue sieves for obtaining viable, single cell suspensions from tissue samples.

### BACKGROUND OF THE INVENTION

In order for tissue culture experiments or studies to be performed, the targeted tissue must be disrupted and broken down to yield viable, single cell suspensions. Currently, mechanical disruption through a wire mesh screen is used in the presence of digestive enzymes such as collagenase to produce whole cell suspensions. One such commercially available wire mesh device is the CELLECTOR tissue sieve. The CELLECTOR device includes a 85 ml or 130 ml pan with a removable mesh screen. In this device, samples are coarsely chopped and typically incubated with an enzymatic solution for a period of time. The tissue sections are then placed into the pan with the appropriate screen. Using a provided pestle, the tissue is then buttered or pushed through the mesh, which is then rinsed with a buffered solution. The filtrate is collected for further culturing of the cell suspension.

Currently available protocols using wire mesh tissue sieves like the CELLECTOR are not well suited when small amounts of tissue are processed. This is due to the fact that the use of digestive enzymes results in lower cell viabilities. In addition, considerable skill is needed to grind the tissue in the relatively large area of mesh screen using the pestle. For example, with respect to the CELLECTOR device, the instructions direct the user to vary the angle and direction of the pestle each time it passes over the sample.

There thus is a need for a device and method that can form viable, single cell suspensions from relatively small tissue samples. In addition, there is a need for a device and method that can produce viable, single cell suspensions without the need of digestive enzymes.

### SUMMARY OF THE INVENTION

In a first aspect of the invention, a syringe tissue sieve for producing viable, single cell suspensions comprises a syringe barrel having a lumen between a proximal end and a distal end thereof. A wire mesh screen is disposed within the lumen of the syringe barrel and is oriented perpendicular to the axial direction of the lumen. The syringe tissue sieve also includes a plunger that is moveable within the lumen of the syringe barrel.

In a second separate aspect of the invention, a syringe tissue sieve includes a syringe barrel having a lumen between a proximal end and a distal end thereof and a collar assembly containing a wire mesh screen, the collar assembly disposed within the lumen of the syringe barrel such the wire mesh screen is oriented perpendicular to the axial direction of the lumen, and a plunger moveable within the lumen of the syringe barrel.

In still another aspect of the invention, a method of producing a viable, single cell suspension from a tissue sample comprises the steps of providing a syringe tissue sieve comprising a syringe barrel having a lumen between a proximal end and a distal end thereof, a wire mesh screen disposed within the lumen of the syringe barrel, the wire mesh screen being disposed perpendicular to the axial direction of the lumen, and a plunger moveable within the lumen of the syringe barrel. The syringe tissue sieve is loaded with a tissue sample and the plunger is inserted into the syringe barrel. The plunger is then repeatedly moved back and forth inside the syringe barrel to produce the viable, single cell suspension.

It is an object of the invention to provide a device and method that can quickly and inexpensively produce a viable, single cell suspension from a tissue sample. It is another object of the invention to provide a device and method which can produce a viable, single cell suspension from a tissue sample that does not require specialized skills or techniques. It is still another object of the invention to provide a device and method that is able to produce a viable, single cell suspension from a tissue sample without the need of digestive enzymes. If digestive enzymes are used in connection with the device and method according to the present invention, the single cell suspension can be produced with less exposure time to the digestive enzymes.

### BRIEF DESCRIPTION OF THE DRAWINGS

FIG. 1 illustrates a syringe tissue sieve according to a preferred embodiment of the invention.

FIG. 2 illustrates an exploded view of a collar assembly that is used to hold in place a wire mesh screen according to another aspect of the invention.

### DETAILED DESCRIPTION OF THE PREFERRED EMBODIMENTS

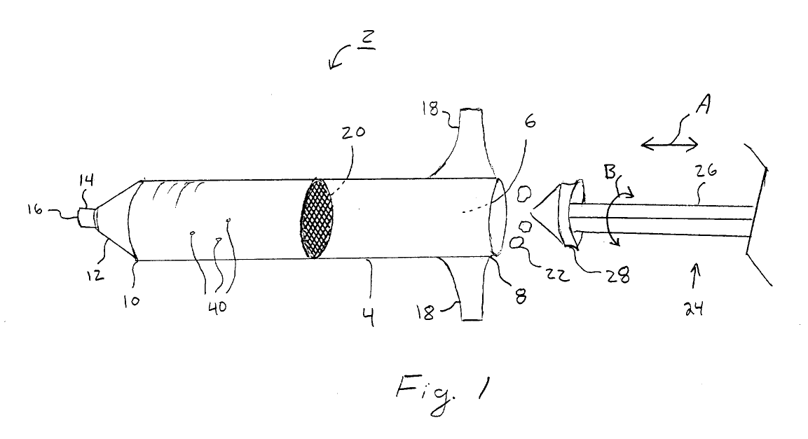

FIG. 1 illustrates a syringe tissue sieve 2 that includes a syringe barrel 4 having a lumen 6 therein between a proximal end 8 and a distal end 10. The distal end 10 preferably includes a tapered end 12 that terminates at an open hub 14. The hub 14 located at the distal end 10 of the syringe barrel 4 contains an opening 16 that provides access to the lumen 6 of the syringe barrel 4. The proximal end 8 of the syringe barrel 4 also is open to provide access to the syringe barrel lumen 6. The syringe barrel 4 may have optional finger grips 18 which aid the user during operation of the syringe tissue sieve 2.

Still referring to FIG. 1, a wire mesh screen 20 is securely fixed to syringe barrel 4 inside the syringe barrel lumen 6. The wire mesh screen 20 is preferably formed from a metal such as stainless steel although other non-metals such as nylon mesh may be employed. The wire mesh screen 20 is preferably a relatively fine mesh. In this regard, it is preferable that the mesh have a mesh size in the range of about 30 to about 120 μm. The size of the mesh is dependent upon a number of factors including, for example, the type of tissue that is being loaded into the syringe tissue sieve 2. Spleen tissue, for example, requires a mesh size of around 120 μm.

In this preferred embodiment, the wire mesh screen 20 is permanently affixed to the interior of the syringe barrel 4. The wire mesh screen 20 may be affixed to the interior of the syringe barrel 4 using any number of techniques such as with the use of a glue or cement. Alternatively, wire mesh screen 20 may be affixed to the interior of the syringe barrel 4 using commonly known bonding techniques for affixing metals to plastic materials.

Still referring to FIG. 1, the wire mesh screen 20 is oriented perpendicular to the axial direction (i.e., the direction of arrow A in FIG. 1) of the lumen 6 of the syringe barrel 4. Consequently, the wire mesh screen 20 is oriented perpendicular to the direction of flow of fluid and tissue 22 during operation.

The syringe tissue sieve 2 also includes a plunger 24 that includes a plunger rod 26 and a stopper 28 affixed to the end of the plunger rod 26. The stopper 28 is preferably made from plastic or rubber material and may optionally include a tapered tip as is shown in FIG. 1. It is preferable that the stopper 28 be sufficiently flexible such that when the plunger 24 is depressed, the entire surface area of the wire mesh screen 20 makes contact with the stopper 28. The plunger 24 is moveable in the direction of arrow A within the lumen of the syringe barrel 4.

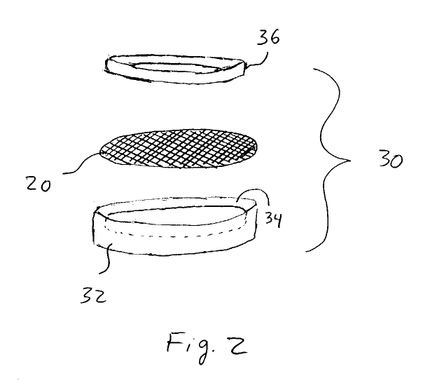

Now referring to FIG. 2, in another preferred embodiment of the invention, the wire mesh screen 20 is secured in a collar assembly 30 that is then loaded into the syringe barrel 4. The collar assembly 30 includes a collar 32 that contains a recessed seat 34 along an inner circumference of the collar 32. The wire mesh screen 20 then rests on this recessed seat 34. The wire mesh screen 20 is held in place by a locking ring 36 that fits on top of the wire mesh screen 20 and within the recessed seat 34 of the collar 32. The locking ring 36 may be secured to the collar 32 simply by a press-fit or it may be thermally bonded. Alternatively, the locking ring 36 may be bonded using a glue or cement. As yet another alternative, the wire mesh screen 20 may be affixed directly to the collar 32. In this case, it the locking ring 36 would not be needed.

The advantage of affixing the wire mesh screen 20 to a collar assembly 30 is that the device may use off-the-shelf syringes. In this regard, the fully constructed collar assembly 30 (with wire mesh screen 20) can be inserted into the syringe barrel 4 of standard syringes. In this aspect of the invention, the collar assembly 30 is constructed to have an outer diameter that is substantially equal to the inner diameter of a standard syringe. Substantially in this context is meant to indicate that the collar assembly 30 is able to snugly fit within the syringe barrel 4 during operation of the device. Finally, in this embodiment, the collar assembly 30 may be designed to be reusable such that the wire mesh screen 20 can be used repeatedly in different syringes.

With reference now to FIGS. 1 and 2, a description of the operation of the syringe tissue sieve 2 will now be given. A tissue sample 22 is obtained and may comprise normal healthy tissue or diseased tissue (e.g., cancerous tissue) depending on the application. The tissue sample 22 is preferably cut into relatively small pieces (about 0.5 g to about 1 g pieces) using a scissors or other sharp instrument. The tissue sample 22 is then loaded into the syringe barrel 4. If the syringe tissue sieve 2 does not use a collar assembly 30, the tissue sample 22 is simply deposited directly into the syringe barrel 4. If the syringe tissue sieve 2 does use a collar assembly 30, prior to loading the tissue sample 22, the user inserts the collar assembly 30 (containing the wire mesh screen 20) into the syringe barrel 4. The tissue sample 22 is then loaded into the lumen 6 of the syringe barrel 4.

The tissue sample 22 may be loaded either dry or with a carrying fluid which may be a buffer, saline solution, or buffer media. After loading of the tissue sample 22, the plunger 24 is inserted into the syringe barrel 4. If the tissue sample 22 was loaded without any carrying fluid, the operator would then fill carrying fluid into the syringe barrel 4 by placing the hub 14 into a fluid solution and withdrawing the plunger 24 in the proximal direction.

Once fluid and sample tissue 22 is located between the wire mesh screen 20 and the stopper 28 of the plunger 24, the operator then repeatedly moves the plunger 24 back and forth inside the syringe barrel 4 in the direction of arrow A. Preferably, when the plunger 24 is depressed towards the distal end 10 of the syringe tissue sieve 2, the stopper 28 is firmly pressed against the surface of the wire mesh screen 20 to thereby sandwich the sample tissue 22 against the wire mesh screen 20. This aids in forming the suspension of single cells 40 that are then located on the other side of the wire mesh screen 20 towards the distal end 10 of the syringe tissue sieve 2. The operator may also rotate the plunger 24 in the direction of arrow B to assist in breaking up the sample tissue 22.

Typically, the plunger 24 must be moved back and forth within the syringe barrel 4 about three to five times. Of course, the number of times the plunger 24 is moved back and forth depends on a number of factors including, for example, the type of sample tissue 22 as well as the size of the sample tissue 22 pieces that are loaded into the syringe tissue sieve 2.

If the syringe tissue sieve 2 uses a removable collar assembly 30, the collar assembly 30 may be optionally removed from the syringe barrel 4. The collar assembly 30 can then be cleaned and sterilized for subsequent use. Of course, it is optional to reuse the collar assembly 30 and the entire syringe tissue sieve 2 may be disposed of after use to avoid any potential for contamination.

As described above, a viable, single cell suspension is created by use of the syringe tissue sieve 2 without the aid of digestive enzymes. However, the sample tissue 22 may be exposed to one or more digestive enzymes such as, for example, collagenase to aid the mechanical sieving process taking place within the syringe barrel 4.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the spirit and scope of the appended claims.

What is claimed is:

1. A syringe tissue sieve for producing viable, single cell suspensions comprising:
a syringe barrel having a lumen between a proximal end and a distal end thereof;
a wire mesh screen disposed within the lumen of the syringe barrel, the wire mesh screen oriented perpendicular to the axial direction of the lumen; and
a plunger moveable within the lumen of the syringe barrel.

2. The syringe plunger of claim 1, wherein the wire mesh screen is between about 30 and about 120 mesh.

3. The syringe plunger of claim 1, wherein the wire mesh screen is removable from the syringe barrel.

4. A syringe tissue sieve for producing viable, single cell suspensions comprising:
a syringe barrel having a lumen between a proximal end and a distal end thereof;
a collar assembly containing a wire mesh screen, the collar assembly disposed within the lumen of the syringe barrel such that the wire mesh screen is oriented perpendicular to the axial direction of the lumen; and
a plunger moveable within the lumen of the syringe barrel.

5. The syringe tissue sieve of claim 4, the collar assembly comprising a collar having a seat and a locking ring.

6. The syringe tissue sieve of claim 4, wherein the collar assembly is removable from the syringe barrel.

7. A method of producing a viable, single cell suspension from a tissue sample comprising the steps of:
providing a syringe tissue sieve comprising a syringe barrel having a lumen between a proximal end and a distal end thereof, a wire mesh screen disposed within the lumen of the syringe barrel, the wire mesh screen being disposed perpendicular to the axial direction of the lumen, and a plunger moveable within the lumen of the syringe barrel;
loading the syringe tissue sieve with a tissue sample;
inserting the plunger into the syringe barrel; and
repeatedly moving the plunger back and forth inside the syringe barrel.

8. The method of claim 7, further comprising the step of exposing the tissue sample to a digestive enzyme.

9. The method of claim 7, further comprising the step of loading the wire mesh screen into the syringe barrel lumen, said step occurring prior to the step of loading a tissue sample into the syringe tissue sieve.

10. The method of claim 7, further comprising the step of removing the wire mesh screen from the syringe barrel lumen, said step occurring after the step of repeatedly moving the plunger back and forth inside the syringe barrel.
